(19) United States
(12) Patent Application Publication
(10) Pub. No.: US 2012/0149884 A1
(43) Pub. Date: Jun. 14, 2012

(54) SPECIFIC ANTIBODY SELECTION BY SELECTIVE ELUTION CONDITIONS

(75) Inventors: Chunmei Guo, Woodbury, MN (US); Patrick A. Mach, Shorewood, MN (US)

(73) Assignee: 3M Innovative Properties Company

(21) Appl. No.: 11/943,168

(22) Filed: Nov. 20, 2007

Related U.S. Application Data
(60) Provisional application No. 60/867,089, filed on Nov. 22, 2006.

(51) Int. Cl. C07K 16/12; C07K 1/22

(52) U.S. Cl. 530/389.5; 530/387.1

(57) ABSTRACT

Methods for preparing high avidity anti-antigen polyclonal antibody preparations by antigen affinity column purification and high avidity anti-antigen polyclonal antibody preparations are described.

### CROSS-REFERENCE TO RELATED APPLICATIONS

The present application claims priority to U.S. Provisional Patent Application Ser. No. 60/867,089, Nov. 22, 2006, which is incorporated herein by reference.

### GOVERNMENT RIGHTS

The U.S. Government may have certain rights to this invention under the terms of DAAD 13-03-C-0047 granted by the Department of Defense.

### SUMMARY OF THE INVENTION

The present invention includes a high avididty anti-Staphylococcus aureus clumping factor protein polyclonal antibody preparation having an avidity expressed as an endpoint concentration of at least 0.01 ng/mL and no greater than 15 ng/mL.

The present invention includes a high avidity anti-Staphylococcus aureus clumping factor protein polyclonal antibody preparation that detects recombinant clumping factor (rClf40) protein of S. aureus at a concentration of about 1 to about 100 picograms per milliliter (pg/mL) in a sample, and more preferably up to about 100 pg/mL in a sample.

The present invention includes a high avidity anti-Staphylococcus aureus clumping factor protein polyclonal antibody preparation that demonstrates at least a 4-fold increase in avidity as measured by endpoint concentration in comparison to a Staphylococcus aureus clumping factor protein antiserum.

The present invention includes a high avidity anti-Staphylococcus aureus clumping factor protein polyclonal antibody preparation as described herein.

The present invention also includes a high avidity anti-Staphylococcus aureus clumping factor protein polyclonal antibody preparation, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to a S. aureus clumping factor (Clf40) protein affinity column; washing the column with a wash buffer having about 0.5 M salt and a pH of about 4; and eluting the high avidity anti-S. aureus clumping factor protein polyclonal antibody preparation from the column with an elution buffer with a pH of about 2. In some embodiments, the high avidity anti-Staphylococcus aureus clumping factor protein polyclonal antibody preparation may be obtained by a method that further includes enriching the antiserum for the IgG class of antibodies prior to binding the antiserum to a S. aureus clumping factor (Clf40) protein affinity column. In some embodiments, enriching for the IgG class of antibodies is by Protein A binding. In some embodiments, the binding, washing, and/or eluting take place in the absence of acetonitrile.

The present invention includes a high avidity anti-Staphylococcus aureus clumping factor protein polyclonal antibody preparation, wherein the high avidity anti-S. aureus clumping factor protein polyclonal antibody preparation is prepared by a method described herein.

The present invention includes a high avidity anti-Staphylococcus aureus clumping factor protein polyclonal antibody preparation that binds to recombinant clumping factor (rClf40) protein of S. aureus with an avidity similar to that of fraction C1 or C2 of experiment B; fraction C of experiment C; fraction C of experiment D; fraction C of experiment E; fraction C of experiment F; fraction C of experiment G; or fraction C of experiment H, as shown in Table 4.

The present invention also includes a high avidity anti-penicillin-binding protein 2a (PBP2a) polyclonal antibody preparation as described herein.

The present invention also includes a high avidity anti-penicillin-binding protein 2a (PBP2a) polyclonal antibody preparation, wherein the high avidity anti-PBP2a polyclonal antibody preparation is prepared by a method described herein. Preferably, the present invention includes a high avidity anti-penicillin-binding protein 2a (PBP2a) polyclonal antibody preparation, wherein the high avidity anti-S. aureus clumping factor protein polyclonal antibody preparation demonstrates at least a 2-fold increase in avidity as measured by endpoint concentration in comparison to a Staphylococcus aureus clumping factor protein antiserum.

The present invention also includes a method of preparing a high avidity anti-Staphylococcus aureus clumping factor protein polyclonal antibody preparation, the method including obtaining antiserum from an animal immunized with recombinant clumping factor (rClf40) protein of S. aureus; binding the antiserum to a S. aureus clumping factor (Clf40) protein affinity column; washing the column with a wash buffer having about 0.5 M salt and a pH of about 4; and eluting the high avidity anti-S. aureus clumping factor protein polyclonal antibody preparation from the column with an elution buffer with a pH of about 2.

Also included in the present invention is a method of preparing a high avidity anti-penicillin-binding protein 2a (PBP2a) polyclonal antibody preparation, the method including obtaining antiserum from an animal immunized with PBP2a; binding the antiserum to a PBP2a affinity column; washing the column with a wash buffer having about 0.5 M salt and a pH of about 4; and eluting the high avidity anti-PBP2a polyclonal antibody preparation from the column with an elution buffer with a pH of about 2.

The present invention includes a method of preparing a high avidity anti-antigen polyclonal antibody preparation, wherein the method includes obtaining an antiserum from an animal immunized with the antigen; binding the antiserum to an antigen affinity column; washing the column with a wash buffer having about 0.5M salt and a pH of about 4; and eluting the high avidity anti-antigen polyclonal antibody preparation from the column with an elution buffer with a pH of about 2.

In some embodiments of the methods of the present invention, the method further includes enriching the antiserum for the IgG class of antibodies prior to binding the antiserum to the antigen affinity column. In some embodiments of the methods of the present invention, enriching for the IgG class of antibodies is by Protein A binding.

In some embodiments of the methods of the present invention, the binding, washing, and/or eluting take place in the absence of acetonitrile.

Unless otherwise specified, “a,” “an,” “the,” and “at least one” are used interchangeably and mean one or more than one.

### BRIEF DESCRIPTION OF THE DRAWINGS

FIG. 1 is a representative chromatogram, illustrating the purified anti-Clf40 antibody protein eluting around 41 minutes.

FIG. 2 shows the DNA sequence for a recombinant plasmid expression vector encoding a fragment of the Staphylococcus aureus mecA protein (SEQ ID NO:1).

### DETAILED DESCRIPTION OF ILLUSTRATIVE EMBODIMENTS OF THE INVENTION

The present invention provides a simple and broadly applicable method for using antigen affinity immunoadsorption and low pH elution for the preparation of polyclonal antibodies with enhanced avidity. The method includes obtaining an antiserum from an animal immunized with the antigen; binding the antiserum to an antigen affinity column; washing the column with a wash buffer including about 0.5M salt and a pH of about 4; and eluting the high avidity anti-antigen polyclonal antibody preparation from the column with an elution buffer with a pH of about 2. Additional purification and/or concentration steps may be performed before and/or after any of the steps of binding the antiserum to an antigen affinity column, washing the column with an elution buffer, or eluting the high avidity anti-antigen polyclonal antibody preparation from the column.

With the present invention, antigen affinity immunoadsorption may be carried out by any of a variety of means. For example, antigen affinity immunoadsorption may be carried out by antigen affinity column chromatography. Column chromatography may be carried out by any mechanical means, for example, carried out in a column run with or without pressure, carried out in a column run from top to bottom or bottom to top, or the direction of the flow of fluid in the column may be reversed during the chromatography process.

Alternatively, antigen affinity immunoadsorption may be carried out by means other than column chromatography. For example, affinity immunoadsorption may be carried out using a batch process in which the solid support is separated from the liquid used to load, wash, and elute the sample by any suitable means, including gravity, centrifugation, or filtration. Affinity immunoadsorption also may be carried out by contacting the sample with a filter that adsorbs or retains some molecules in the sample more strongly than others.

The antigen affinity column may be prepared by any of a variety of methods, including, but not limited to, those described herein. The binding of the antiserum to an antigen affinity column may be carried out by any of a wide variety of immunoadsorption methods, including, but not limited to, those described herein. The binding of the antiserum to the antigen affinity column may occur in a variety of buffers or salts including, but not limited to, sodium, potassium, ammonium, chloride, acetate, phosphate, citrate, Tris buffers and/or organic buffers with a buffering capacity near neutrality. Specific examples of such buffers and salts include, for example, Tris, sodium phosphate, potassium phosphate, ammonium phosphate, sodium chloride, potassium chloride, ammonium chloride, sodium citrate, potassium citrate, ammonium citrate, sodium acetate, potassium acetate, or ammonium acetate.

With the methods of the present invention, the immunoadsorption column is washed with a wash buffer having about 0.5 M salt and a pH of about 4. This buffer with a pH of about 4 interferes with the binding of low avidity anti-antigen antibodies to the antigen column. Such low avidity antibodies are, thus, eluted from the column. Suitable elution buffers with a pH of about 4 include, but are not limited to, phosphate buffers, Tris buffers, acetate buffers, and/or citrate buffers. Suitable salts include, but are not limited to, sodium chloride, potassium chloride, ammonium chloride, sodium acetate, potassium acetate, and/or ammonium acetate. Other buffers and salts can be used. In a preferred embodiment, the wash buffer is 20 mM sodium acetate, 0.5M NaCl, pH 4. Suitable wash buffers with a pH of about 4 include, for example, buffers with a pH of about 3 to about 5; buffers with a pH of about 3.1 to about 4.9; buffers with a pH of about 3.2 to about 4.8; buffers with a pH of about 3.3 to about 4.7; buffers with a pH of about 3.4 to about 4.6; buffers with a pH of about 3.5 to about 4.5; buffers with a pH of about 3.6 to about 4.4; buffers with a pH of about 3.7 to about 4.3; buffers with a pH of about 3.8 to about 4.2; or buffers with a pH of about 3.9 to about 4.1. Suitable buffers with a pH of about 4 include, for example, buffers of about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8 about 4.9, about 5, about 5.5, about 6, or about 6.5. Suitable buffers with a pH of about 4 include buffers with a pH range with an upper limit and a lower limit selected from any of these listed pHs.

With the methods of the present invention, the high avidity anti-antigen polyclonal antibody preparation is eluted from the immunoadsorption column with an elution buffer with a pH of about 2. Suitable elution buffers with a pH of about 2 include, but are not limited to, phosphate buffers, Tris buffers, acetate buffers, and/or citrate buffers. Suitable elution buffers may include acetic acid, glycine, or citric acid. Suitable elution buffers with a pH of about 2 include, for example, elution buffers with a pH of about 1.5 to about 3.0; elution buffers with a pH of about 1.5 to about 2.9; elution buffers with a pH of about 1.5 to about 2.8; or elution buffers with a pH of about 1.9 to about 2.1. Suitable elution buffers with a pH of about 2 include, for example, elution buffers with a pH of about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 2.95, or about 3. Suitable elution buffers with a pH of about 2 include buffers with a pH range with an upper limit and a lower limit selected from any of these listed pHs. In a preferred embodiment, the elution buffer with a pH of about 2 is 0.1M citric acid, pH 2.1.

Elution with an elution buffer with a pH of about 2 releases high avidity antibodies that have been bound to the antigen affinity column, resulting in the isolation of a high avidity anti-antigen polyclonal antibody preparation. Elution with an elution buffer with a pH of about 2 effectively enriches the polyclonal antibody preparation for those antibodies having the greatest affinity for the target antigen.

Thus, the methods of present invention permit the isolation of a high avidity anti-Staphylococcus aureus clumping factor protein polyclonal antibody preparation. Avidity of a polyclonal antibody preparation is expressed as an endpoint titer concentration. As used herein, “endpoint titer concentration” is the minimum concentration of the polyclonal antibody preparation required to generate a positive response in an Enzyme-Linked Immunosorbant Assay (ELISA). Thus, greater avidity of a polyclonal antibody preparation is indicated when a polyclonal antibody preparation generates a positive ELISA response at a lower endpoint concentration. A positive response in an ELISA may be determined by any suitable standard. For example, a positive ELISA response may include the generation of a signal that is equal to or greater than three standard deviations above a background signal. As another example, a positive ELISA response may be indicated when the signal generated by the ELISA produces an absorbance at 405 nm (A₄₀₅) using a spectrophotometric plate reader.

Certain high avidity polyclonal antibody preparations can have an avidity expressed as an endpoint concentration of, for example, from about 0.01 nanograms per milliliter (ng/mL) to about 15 ng/mL, although in certain embodiments, a high avidity polyclonal antibody preparation of the present invention can have an avidity expressed as an endpoint concentration outside of this range. A high avidity polyclonal antibody preparation can have an avidity expressed as an endpoint concentration of, for example, at least 0.313 ng/mL such as, for example, at least 0.5 ng/mL, at least 0.625 ng/mL, at least 1.0 ng/mL, at least 1.25 ng/mL, at least 2.0 ng/mL, at least 3.9 ng/mL, or at least 7.8 ng/mL. A high avidity polyclonal antibody preparation can have an avidity expressed as an endpoint concentration of no greater than 15 ng/mL such as, for example, no greater than 12 ng/mL, no greater than 10 ng/mL, no greater than 7.8 ng/mL, no greater than 3.9 ng/mL, or no greater than 2.0 ng/mL. A high avidity polyclonal antibody preparation can have an avidity expressed as an endpoint concentration in a range defined by endpoints selected from any of these listed endpoint concentrations such as, for example, avidity expressed as an endpoint concentration ranging from, for example, 0.5 ng/mL to 15 ng/mL, 0.625 ng/mL to 7.8 ng/mL, 0.5 ng/mL to 3.9 ng/mL, 3.9 ng/mL to 7.8 ng/mL, and the like.

Protein and antibody concentrations in a sample at any stage of purification can be determined by any suitable method. Such methods are well known in the art and include, but are not limited to colorimetric methods such as the Lowry assay, the Bradford assay, the Smith assay, and the colloidal gold assay; methods utilizing the UV absorption properties of proteins; and visual estimation based on stained protein bands on gels relying on comparison with protein standards of known quantity on the same gel.

As used herein, antiserum refers to the blood from an immunized host animal from which the clotting proteins and red blood cells (RBCs) have been removed. An antiserum (also referred to herein as an “antiserum preparation,” “crude antiserum,” or “raw antiserum”) still possesses immunoglobulins of all classes as well as other various serum proteins. Thus, in addition to antibodies that recognize the target antigen, the antiserum also contains antibodies to various non-target antigens that can sometimes react non-specifically in immunological assays.

An antiserum to a target antigen may be obtained by immunizing any of a variety of host animals. Any of a wide variety of immunization protocols may be used. The host animal may be any mammal, for example, a mouse, hamster, rat, rabbit, guinea pig, goat, sheep, horse, cow, buffalo, bison, camel, or llama. A host animal may be a bird, for example, a chicken or a turkey. In some embodiments, an antiserum preparation, rather than obtained from as a blood sample, is obtained from another fluid source, for example, from milk, colostrums, egg white, or egg yolk. In some embodiments, an antiserum preparation is obtained, not by immunizing a host animal with the target antigen, but rather, from an individual with a prior exposure to the antigen or from pooled serum, for example, from pooled human serum.

Any of a wide variety of target antigens may be used in the methods of the present invention, including, but not limited to, microbes, including, for example, bacteria, viruses, yeast, and fungi, proteins, peptides, carbohydrates and combinations thereof. Proteins and peptides may be, for example, naturally occurring, chemically synthesized, or recombinantly produced. An antigen may be conjugated to a carrier. In preferred embodiments, the target antigen is the recombinant clumping factor fragment (rClf40) (Inhibitex, Inc., Alpharetta, Ga.), penicillin-binding protein 2a (PBP2a), or S. aureus Protein A.

In some embodiments of the present invention, a raw antiserum preparation may be enriched prior to binding the antiserum to an antigen affinity column. Such enrichment may eliminate non-immunoglobulin proteins from the preparation and/or enrich for one or more classes of immunoglobulin (e.g., IgG) within the sample. Any of a variety of methods may be used to obtain such an enriched antiserum preparation, including, but not limited to, those described herein. Methods of eliminating non-immunoglobulin serum proteins from an antiserum preparation and methods for enriching for the IgG fraction are well known in the art. For example, ammonium sulfate precipitation, Protein A binding, Protein G binding, or caprylic acid precipitation may be used to enrich for the IgG class of antibodies.

A population of monoclonal antibodies is homogeneous. All of the monoclonal antibodies in the preparation recognize the same epitope on the target molecule and all of the monoclonal antibodies have the same affinity. As used herein, affinity is the binding strength of the interaction of a monoclonal antibody with its antigenic epitope. As used herein, an epitope is the portion of an antigen bound by an antibody. The higher the affinity, the tighter the association between antigen and antibody, and the more likely the antigen is to remain in the binding site.

In contrast, polyclonal antibodies are heterogeneous. Polyclonal antibodies are not derived from a single clone, and therefore have variations in structure and in their binding mechanism. Even though they all are capable of binding the target antigen, they represent a pooled mixture of antibodies derived from many different clones, each with a unique affinity for the target antigen. Thus, polyclonal antibodies can recognize different epitopes on the target antigen and various antibodies within the pooled mixture can have different binding affinities for the target antigen. Because the heterogeneous population of polyclonal antibodies binds to the antigen target at different epitopes, with different affinities, the overall efficiency of target binding can be synergistically increased.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀). K₀ is the value of K when the ligand concentration is such that one-half of the antigen-binding sites are filled. Antibody avidity may be measured using methods known in the art which assess degree of binding of antibody to antigen. These methods include competition assays and non-competition assays.

Polyclonal antibody preparations may be further evaluated to determine quality. For example, the protein concentration of specific antibody in a preparation may be determined For example, the protein concentration of specific antibody in a purified fraction relative to the protein concentration of total IgG in the preparation prior to fractionation may be determined.

In some embodiments of the method, the binding, washing, and/or eluting take place in the absence of acetonitrile.

The present invention includes high avidity polyclonal antibody preparations prepared by the methods described herein.

The present invention also includes high avidity polyclonal antibody preparations with antigen binding characteristic that are similar to or the same as the antigen binding characteristics of the high avidity polyclonal antibody preparations prepared by the methods described herein.

High avidity anti-antigen polyclonal antibody preparations of the present invention may detect target antigen at concentrations of at least about 1 picogram per milliliter (pg/mL) in a sample, and more preferably up to about 100 pg/mL in a sample. High avidity anti-antigen polyclonal antibody preparations of the present invention may detect target antigen at concentrations of less than about 100 pg/mL in a sample. High avidity anti-antigen polyclonal antibody preparations of the present invention may detect target antigen at concentrations of less than about 50 pg/mL in a sample. High avidity anti-antigen polyclonal antibody preparations of the present invention may detect target antigen at concentrations of less than about 2 pg/mL in a sample.

High avidity anti-antigen polyclonal antibody preparations of the present invention may demonstrate at least a 2-fold increase, at least a 4-fold increase, at least an 8-fold increase, at least a 10-fold increase, at least a 15-fold increase, at least a 20-fold increase, at least a 25-fold increase, at least a 30-fold increase, at least a 40-fold increase, at least a 50-fold increase, at least a 60-fold increase, at least a 70-fold increase, at least a 75-fold increase, at least a 80-fold increase, at least a 90-fold increase, or at least a 100-fold increase in avidity as measured by endpoint concentration in comparison to a raw antiserum or to an IgG enriched antiserum preparation.

A preferred high avidity polyclonal antibody preparation of the present invention may be, but is not limited to, a high avidity polyclonal antibody preparation that binds to rClf40 with antigen binding characteristics that are similar to or the same as the antigen binding characteristics of the high avidity polyclonal antibody preparations described in the Examples included herewith and high avidity polyclonal antibody preparations that bind to PBP-2a with antigen binding characteristics that are similar to or the same as the antigen binding characteristics of the high avidity polyclonal antibody preparations described in the Examples included herein.

Such a high avidity anti-rClf40 polyclonal antibody preparation may bind to recombinant clumping factor (rClf40) protein of S. aureus with antigen binding activity similar to that of fraction C1 or C2 of experiment B; fraction C of experiment C; fraction C of experiment D; fraction C of experiment E; fraction C of experiment F; fraction C of experiment G; or fraction C of experiment H, as shown in Table 4.

The high avidity anti-target polyclonal antibody preparations of the present invention demonstrate an improved capability to capture target antigen from solution. The antibodies are highly specific for the target antigen. Because they are polyclonal in nature, the antibodies recognize a variety of epitopes on the antigen. Coupled with the high specificity for the antigen, the binding of the antibodies to various epitopes increases the overall antigen binding capacity of the pool of high affinity antibodies. The high avidity anti-target polyclonal antibody preparations of the present invention will be useful, for example, in improved immunoassays.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Purification of High-Affinity Anti-Clumping Factor from Rabbit Antisera

### A. Preparation of Polyvalent Anti-Clf40 Antisera.

Pooled rabbit serum was used for the purification of anti-Clf40 antibodies. The rabbits were immunized with the recombinant Clumping Factor (rClf40, Inhibitex, Inc., Alpharetta, Ga.) according the schedule shown in Table 1. The dose of the antigen (rClf40) in each immunization was 200 μg. All immunizations were administered by subcutaneous injection.

TABLE 1
Immunizaion Protocol

| Day | Activity | Adjuvant |
|---|---|---|
| 1 | Primary | Complete |
| | Injection | Freund's |
| 21 | Boost #1 | Incomplete |
| | | Freund's |
| 35 | Boost #2 | Incomplete |
| | | Freund's |
| 49 | Boost #3 | Incomplete |
| | | Freund's |

Blood samples were drawn, prior to the primary injection and subsequent to the second and third booster injections, for the determination of anti-Clf40 antibody titers in the rabbit serum using an ELISA protocol.

Pooled rabbit anti-Clf40 antisera (RxClf40) were precipitated by drop-wise addition of ice-cold saturated ammonium sulfate to a final ratio of 1:1 (50% saturated ammonium sulfate). This procedure was done in an ice-cold beaker, with constant stirring. The supernatant was transferred to a 50-mL centrifuge tube and placed on a reciprocating mixer overnight at 4° C. The suspension was centrifuged at 10,000×g for 30 minutes at 2° C. The supernatant was removed and the pellet was resuspended in an equal volume of deionized water. The resuspended protein was dialyzed (12,000-14,000 MW cut-off dialysis tubing) against 2 liters (L) of PBS. The dialysis buffer was removed and replaced after about two hours and again after about 24 hours. After about 48 hours of dialysis, the protein dialysate was removed and filtered with a 0.22 μm filter. The filtrate was used for purification of the IgG fraction.

The IgG fraction was recovered from the filtrate by chromatography, using a Protein A affinity column from BioRad Laboratories, Inc. (Hercules, Calif.) according to the manufacturer's instructions. The material recovered from this procedure was the starting material for affinity purification with the Clf40 affinity chromatography column.

### B. Preparation of a Clf40 Affinity Column.

### Affinity Antigen

All buffers and solutions used in this procedure were at about 4° C. prior to use unless noted otherwise. Staphylococcus aureus clumping factor (Clf40) protein, prepared from a recombinant microorganism expressing the ClfA gene (see U.S. Pat. No. 6,177,084), was obtained from Inhibitex, Inc. (Alpharetta, Ga.). The Clf40 protein was suspended in phosphate buffered saline (PBS, 0.15 M NaCl in 0.01 M sodium phosphate, pH 7.0) to a concentration of 2.6 mg/mL. The suspension (12 mL) was dialyzed overnight in 2 L of coupling buffer (0.1 M NaHCO₃, pH 8.3, with 0.5 M NaCl).

### Preparation of Support Material

The NHS activated Sepharose resin (GE HealthCare, Waukesha, Wis.) was used as the support material for the affinity column. Eight milliliters of the resuspended resin was placed at room temperature in a filter funnel with a scintered glass frit. The resin was washed by adding five sequential 200 mL aliquots of 1 mM HCl to the filter funnel, to which a slight vacuum was applied. The entire washing process, after which all of the acid solution was removed by vacuum aspiration, took approximately 15 minutes.

### Coupling Procedure:

All buffers and solutions used in this procedure were at 4° C. prior to use. The coupling procedure was conducted at room temperature. The washed resin was added to the dialyzed Clf40 solution (12 mL) in a 50-mL screw-capped conical tube. The tube was sealed and the mixture was rotated end-over-end overnight at 4° C. The excess (Clf40) ligand was removed by washing the resin with five volumes (25 mL/wash) of coupling buffer. The coupling buffer was aspirated and the resin was transferred to 12 mL 0.1 M Tris-HCl buffer, pH 8.0, for two hours to block any unreacted binding sites on the resin. Subsequently, the resin was washed (40 mL/wash) at room temperature with each of the following solutions: 0.1M sodium acetate buffer, pH 4.0 containing 0.5 M NaCl followed by 0.1 M Tris-HCl, pH 8.0, plus 0.5 M NaCl. After completion of the wash cycles, the resin was packed into the chromatography column (MF-PLUS column, 100 mm×7.5 mm, Alltech Associates, Inc., Deerfield, Ill.) with a syringe pump, using PBS buffer, pH 7.3, as the suspending liquid for column packing.

### C. Chromatography Conditions

A Shimadzu HPLC system (Model SCL-10AVP, Shimadzu Corporation, Shimadzu Scientific Instruments, Inc., Columbia, Md.) was used for all preparative chromatography runs. The solvents used for the binding and elution of the anti-Clf40 antibodies are listed below. All buffers were prepared with deionized water using a MILLI Q filtration system (Millipore Corp., Billerica, Mass.) and were filtered through a 0.22-μm (pore size) membrane filter.

TABLE 2

| SOLVENT | COMPOSITION |
|---|---|
| Binding Buffer A | Phosphate Buffered Saline, pH 7.3 |
| Elution Buffer B | 20 mM Sodium Acetate, 0.5M NaCl, pH 4.0 |
| Elution Buffer C | 0.1M Citric Acid, pH 2.1 |

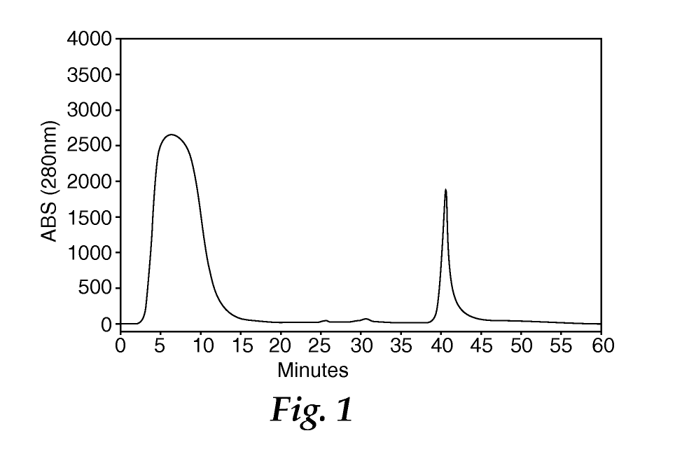

The column was pre-equilibrated with binding buffer A prior to sample injection. The samples of RxClf40 sera were manually injected into the column via a sample injection loop at T=0 minutes. After sample injection, the solvents listed in Table 3 were run through the column at the specified times and flow rates. In some experiments, after the antibody sample was injected onto the column, the column eluate was recycled through the column for two hours prior to switching to Elution Buffer B. Fractions of the column eluate from each of the mobile phase solvents were collected and analyzed for anti-Clf40 antibodies using the ELISA assay described below. A representative chromatogram, illustrating the purified anti-Clf40 antibody protein eluting around 41 minutes, is shown in FIG. 1.

TABLE 3
Mobile phases used for the affinity purification of anti-Clf40 antibodies.
The first column indicates at what time period, after injection, each
mobile phase is run through the column.

| Time | | Flow Rate |
|---|---|---|
| (min) | Solvent | (ml/min) |
| 0-20 | Binding Buffer A | 0.6 |
| 20-35 | Elution Buffer B | 1.0 |
| 35-50 | Elution Buffer C | 1.0 |
| 50-60 | Binding Buffer A | 1.0 |

### D. ELISA Assay for Anti-Clf40 Antibody Activity.

Pooled sera and eluent fractions from the affinity column were screened for the presence of anti-Clf40 antibodies using the following procedure. The 96-well plates (Corning 3361; Corning, Inc. Life Science, Lowell, Mass.) were coated with 1 μg/mL (100 μL/well) Clf40 (Inhibitex, Inc., Alpharetta, Ga.) diluted in PBS. The plates were incubated for one hour at 37° C. Following the incubation, each well was washed five times with 350 μL of PBST (Phosphate buffered saline containing 0.05% (w/v) TWEEN 20) and blocked with Blotto solution (PBST containing 2% (w/v) dry milk, 200 μL/well) for one hour at 37° C. After the blocking process, the wells were washed five times as described above.

Serial two-fold dilutions of rabbit serum samples (or affinity column fractions), diluted in 100 μL PBBT (PBST containing 0.1% Bovine Serum Albumin) were added to duplicate wells in the plates. The plates were incubated approximately one hour at 37° C., followed by five washes, as described above. Alkaline phosphatase-conjugated goat anti-rabbit IgG antibody (0.5 μg/mL in PBBT, Jackson Immunoresearch Laboratories, Inc., West Grove, Pa.) was added to each well (100 mL/well) and the plates were incubated at 37° C. for one hour. After washing the wells as described above, the alkaline phosphatase substrate, p-nitrophenylphosphate (PNPP, KPL, Inc. Gaithersburg, Md.), was added to each well and the plates were held at room temperature for 15 minutes. A stop solution (5% w/v disodium EDTA) was added to each well and the absorbance at 405 nm (A₄₀₅) of each well was measured using a plate reader. The titer of a sample was determined to be the highest dilution that gave a positive response (A₄₀₅>0.1 absorbance units) in the ELISA assay.

### E. Affinity Purification of Anti-Clf40 Antibody

In these experiments, the affinity purification method was used on two different lots of pooled RxClf40 sera. One lot exhibited a high titer for anti-Clf40 antibodies in the ELISA testing; the other lot exhibited a relatively lower titer of anti-Clf40 antibody. The samples were all diluted to 1.75 mg protein/mL in PBS buffer prior to conducting the column chromatography. In these experiments, four different amounts of protein were loaded onto the column: 5 mg, 10 mg, 20 mg, and 40 mg, respectively. This was done by varying the volume of sample injected onto the column (for example, for a 5 mg column load, 2.86 mL of sample were injected, for a 10 mg column load, 5.71 mL of sample were injected).

Table 4 summarizes the results of eight experiments using the affinity purification procedure described above. Various portions, corresponding to the chromatography mobile phase (Table 3) of the column eluate, were collected. The fraction “A-R” corresponds to the column eluate after the sample had been recirculated through the column for two hours. In some experiments, subfractions (“C1” and “C2”, corresponding to approximately the first and second half of the elution buffer volume, respectively) of the citric acid elution buffer were collected and analyzed separately.

With the exception of Experiment A, all of the data indicate that the affinity purification process resulted in an eluate (Fraction C) that was enriched for anti-Clf40 antibody, relative to the IgG starting material. Experiments B-H are representative of additional experiments performed. Experiment A was a preliminary experiment in which the inventors were optimizing the conditions for purification of anti-Clf40 IgG. There is a preponderance of subsequent data, including experiments B-H, showing that the antibody purification results of Experiment A were atypical.

Experiments C and D show that, when the starting material had a relatively low titer of antibody, the affinity purification process resulted in an eluate that was highly enriched for anti-Clf40 antibody. Experiments E-H demonstrate the purification of anti-Clf40 antibody without recirculation of the starting material on the column. Additionally, experiments E-H demonstrate the reproducibility of the affinity purification process.

TABLE 4
Recovery of protein and anti-Clf40 antibody activity
from various fractions of hyperimmune rabbit serum.

| | | | | Total | | |
|---|---|---|---|---|---|---|
| | | | Total Protein | Protein/ | | Anti-Clf |
| | Column | | Recovered | Fraction | % of Total | Titer |
| Experiment | Load | Fraction Buffer | (mg) | (mg) | Protein | (ng/mL) |
| A | | Starting Material | | | | 15 |
| | 5 mg High | A | 5.47 | 0.227 | 4.15 | >500 |
| | Titer Rbt x | A-R | | 2.325 | 42.50 | >500 |
| | ClfA Sera | B | | 0.783 | 14.32 | 125 |
| | Pool | C1 | | 1.186 | 21.67 | 31 |
| | | C2 | | 0.941 | 17.20 | 31 |
| B | | Starting Material | | | | 3.9 |
| | 20 mg High | A | 13.07 | 0.991 | 7.58 | 125 |
| | Titer Rbt x | A-R | | 9.904 | 75.78 | 31 |
| | ClfA Sera | B | | 0.301 | 2.31 | 2 |
| | Pool | C1 | | 1.562 | 11.95 | 0.5 |
| | | C2 | | 0.312 | 2.39 | 1 |
| C | | Starting Material | | | | 500 |
| | 20 mg Low | A | 22.30 | 2.369 | 10.62 | >500 |
| | Titer Rbt x | A-R | | 19.359 | 86.81 | >500 |
| | ClfA Sera | B | | 0.122 | 0.55 | |
| | Pool | C | | 0.450 | 2.02 | 3.9 |
| D | | Starting Material | | | | 500 |
| | 40 mg Low | A | 43.59 | 2.495 | 5.72 | >500 |
| | Titer Rbt x | A-R | | 39.933 | 91.61 | >500 |
| | ClfA Sera | B | | 0.233 | 0.54 | 62 |
| | Pool | C | | 0.926 | 2.12 | 7.8 |
| E | | Starting Material | | | | 15.6 |
| | 10 mg Rb- | A | 8.00 | 6.579 | 82.20 | 250 |
| | CLFA pool | B | | 0.430 | 5.37 | 7.8 |
| | 517.21 A-F | C | | 0.995 | 12.43 | 2 |
| F | | Starting Material | | | | 15.6 |
| | 10 mg Rb- | PBS | 8.16 | 6.886 | 84.43 | 250 |
| | CLFA pool | B | | 0.258 | 3.16 | 15.6 |
| | 517.21 A-F | C | | 1.012 | 12.40 | 2 |
| G | | Starting Material | | | | 15.6 |
| | 10 mg Rb- | A | 8.35 | 6.761 | 80.94 | 250 |
| | CLFA pool | B | | 0.626 | 7.49 | 31 |
| | 517.21 A-F | C | | 0.966 | 11.57 | 2 |
| H | | Starting Material | | | | 15.6 |
| | 10 mg Rb- | A | 8.74 | 6.783 | 77.64 | 250 |
| | CLFA pool | B | | 0.350 | 4.01 | 15.6 |
| | 517.21 A-F | C | | 1.603 | 18.35 | 3.9 |

Protein was determined spectrophotometrically by absorbance at 280 nm and dividing by the extinction coefficient factor of rabbit IgG (i.e., 1.4). Antibody titer was determined according to Example ID. Antibody titers listed in this table were calculated as the average of at least two duplicate wells in the ELISA assay.

### Example 2

### Purification of High-Affinity Penicillin Binding Protein 2a (PBP2a) Antibody from Goat Antisera

### A. Preparation of PBP2a Antigen

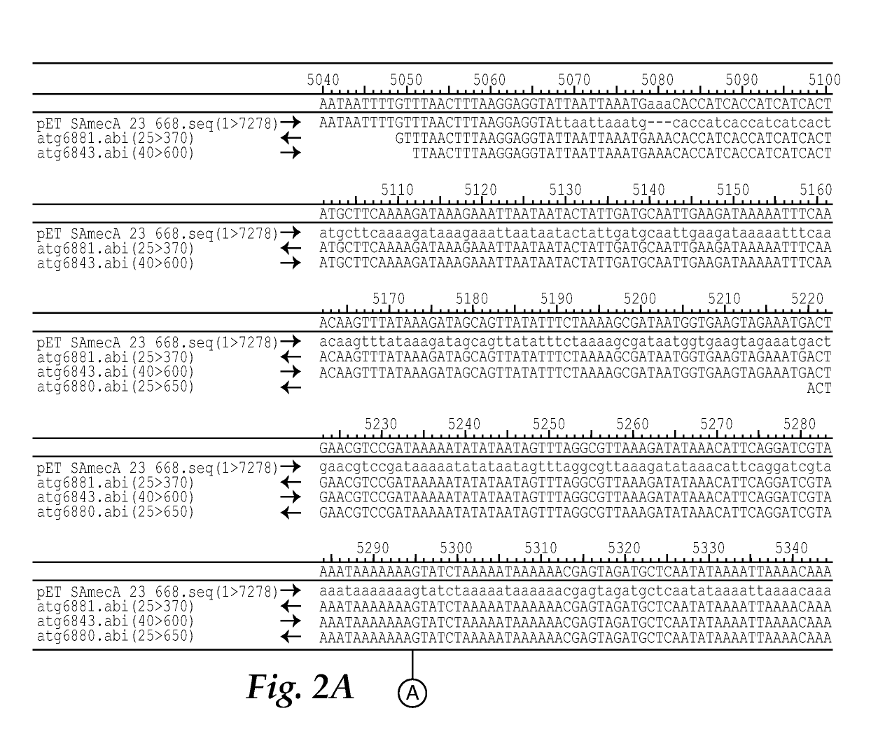

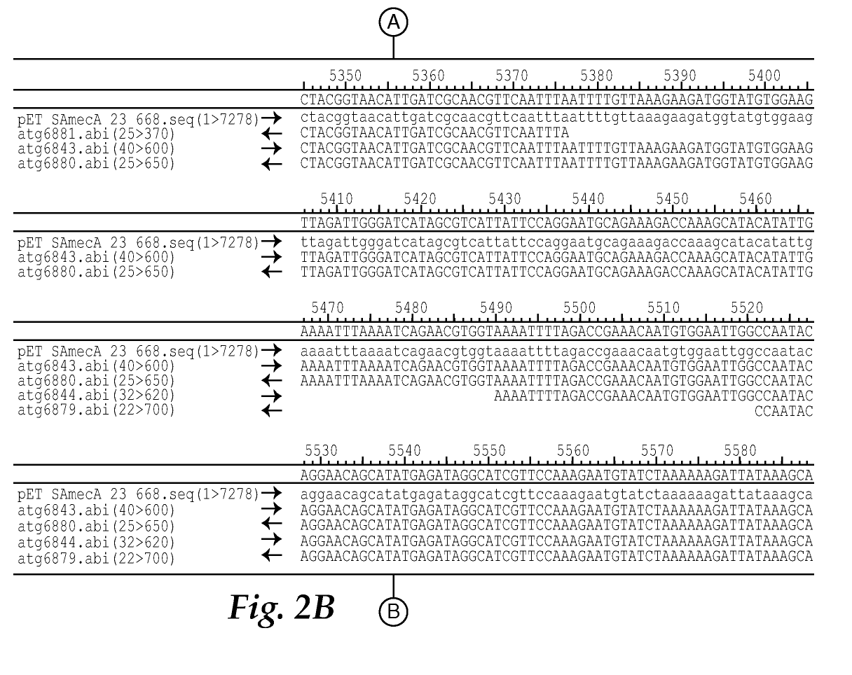

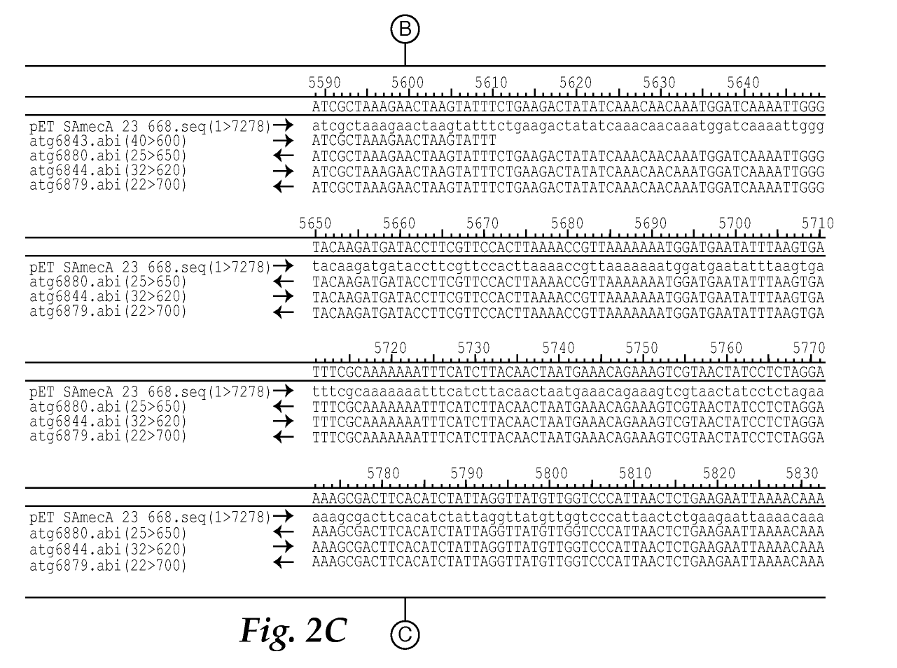

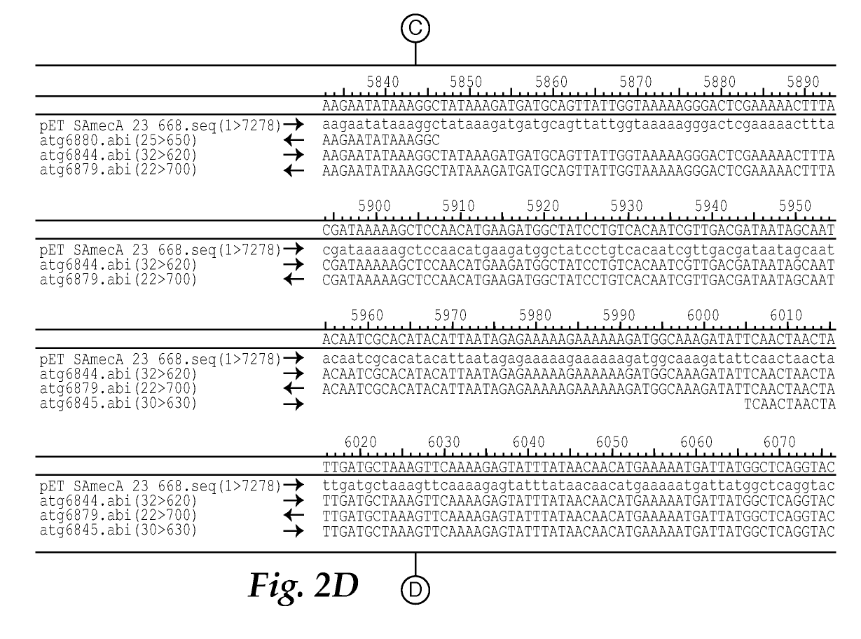

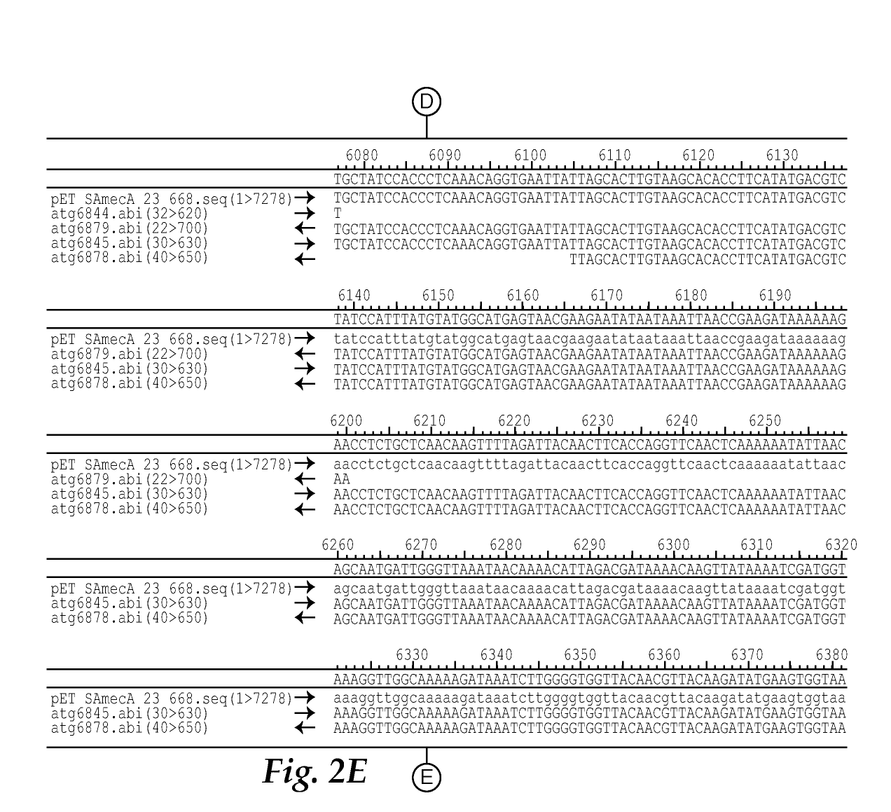

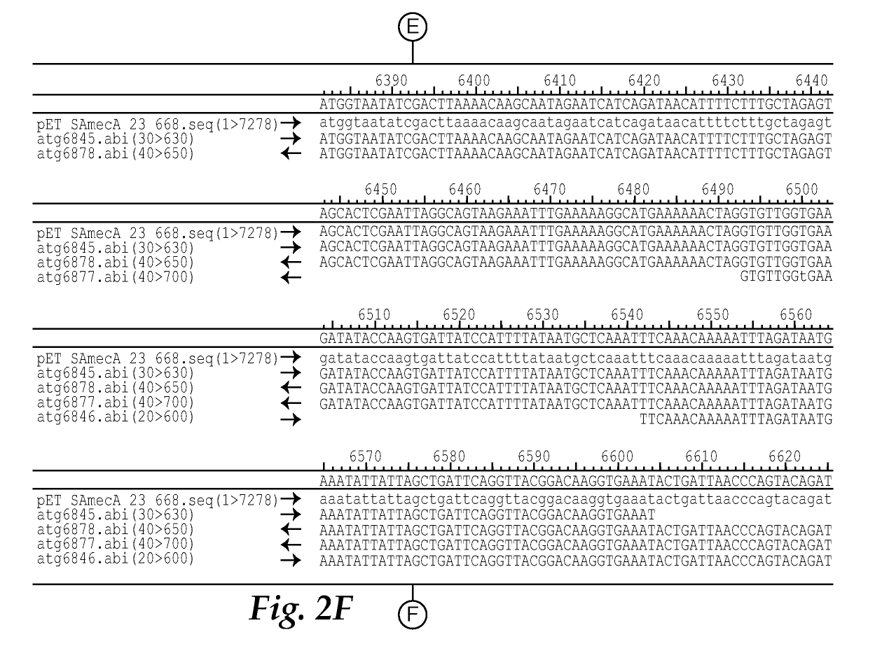

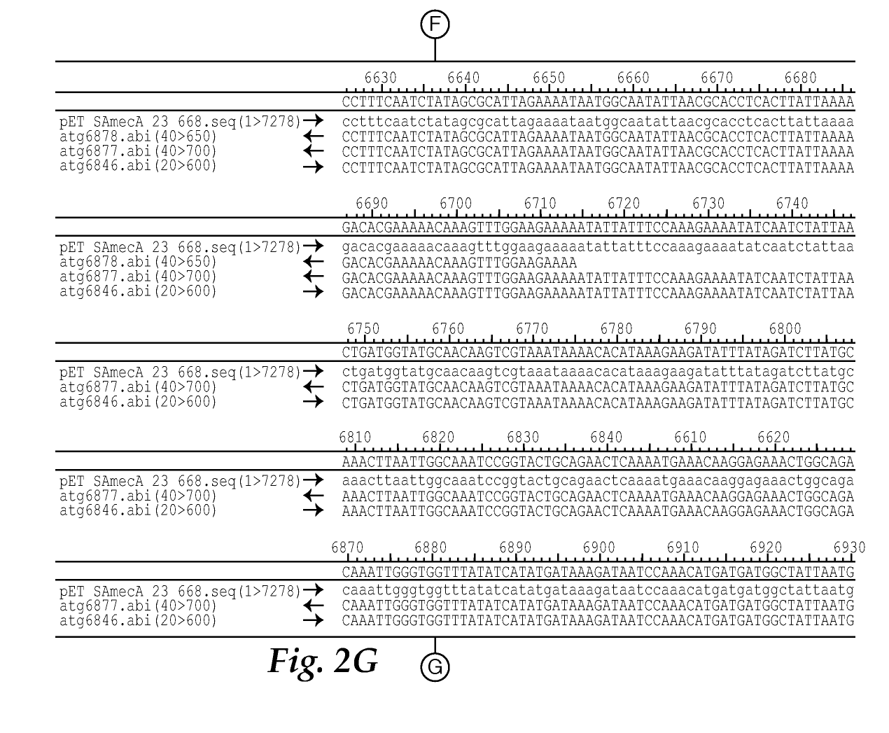

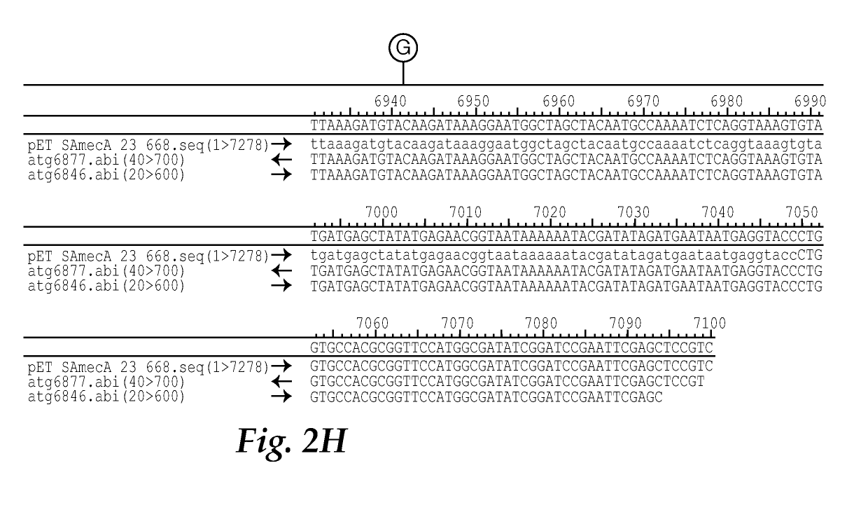

A recombinant plasmid expression vector containing the S. aureus mecA DNA sequence shown in FIG. 2 (SEQ ID NO:1) was constructed by inserting the DNA sequence into the PacI and KpnI restriction endonuclease sites of the Novagen expression vector pET29 (EMD Biosciences, Inc., San Diego, Calif.). The bacterial strain used for the cloning experiments was Escherichia coli DH5-α.

For gene expression experiments, the recombinant plasmid was transformed into the E. coli expression strain, BL21(DE3), which was obtained from Stratagene, Inc. (La Jolla, Calif.). The expression strain was grown and His6-PBP2a-washed inclusion bodies were prepared using the methods of Frank, et al. (“High-Yield Expression, Refolding, and Purification of Penicillin-Binding Protein 2a from Methicillin-Resistant Staphylococcus aureus Strain 27R,” Protein Expression and Purification, volume 6, no. 5, pp. 671-675, 1995). The final pellets of washed inclusion bodies were stored at −80° C.

Washed inclusion body pellets were homogenized (VIRTIS HANDISHEAR, SP Industries, Inc., Warminster, Pa.) in 50 mM Tris(HCl), pH 8.0, 500 mM NaCl, 5 M guanidine, 0.01% thiodiglycol (w/v), and 0.1 mM PMSF (2 tube, 30 mL/tube) and incubated at room temperature for two hours followed by overnight incubation at 4° C. Insoluble material was sedimented at 16,000 rpm for 30 minutes 4° C. The supernatants were removed, pooled in one container, and incubated with 40 mL of Ni-NTA agarose 50% slurry, for 60 minutes at room temperature, rotating slowly. The beads were sedimented, non-bound protein in the supernatant was removed, and the beads were washed twice with 50 mL of 50 mM Tris(HCl) pH 8.0, 500 mM NaCl, 5 M guanidine, 20 mM imidazole, 0.0 1% thiodiglycol (w/v), and 0.1 mM PMSF. Following the second wash, fresh buffer was added so that the total volume of beads and buffer was 40 mL. The mixture was diluted with 160 mL of 50 mM NaH₂PO₄ pH 8, 500 mM NaCl, 20 mM imidazole, 20% glycerol (v/v) 0.01% thiodiglycol (v/v), and 0.1 mM PMSF, added slowly over 10-15 minutes in 5-10 mL aliquots and mixed after each addition. The diluted mixture was rotated slowly at 4° C. for 30 minutes. The beads were sedimented, non-bound protein in the supernatant was removed, and the beads were washed twice with 30 mL of 50 mM NaH₂PO₄, pH 8, 500 mM NaCl, 20 mM imidazole, 20% glycerol (v/v), 0.01% thiodiglycol (v/v), and 0.1 mM PMSF. Specifically bound protein was eluted by washing the beads with 10 mL of 50 mM NaH₂PO₄, pH 8, 500 mM NaCl, 200 mM imidazole, 20% glycerol (v/v) 0.01% thiodiglycol (v/v), and 0.1 mM PMSF four times. The fractions were pooled, filter sterilized, and dialyzed against a solution containing 50 mM NaH₂PO₄, pH 7.2, 500 mM NaCl and 25% (v/v) glycerol. The colorless, clear dialysate was stored in 5 mL aliquots at −80° C. For immunizations, this preparation was diluted directly into the adjuvant prior to injection.

### B. Preparation of Polyvalent Anti-PBP2a Antisera.

Pooled goat serum was used for the purification of anti-PBP2a antibodies. The goats were injected with the antigen in Complete Freund's Adjuvant on day 1. Subsequent injections of the antigen suspended in Incomplete Freund's Adjuvant were administered on days 14, 28, 42, 56, 70, 84, 98, 112, and 119. The dose of the antigen in each immunization was 500 n. All immunizations were administered by subcutaneous injection. The goat serum was harvested for affinity purification on day 120 of the immunization schedule.

Blood samples were drawn prior to the primary injection and subsequent to the second and third booster injections for the determination of anti-PBP2a antibody titers in the goat serum using an ELISA protocol.

Protein impurities in the pooled goat antisera were precipitated with caprylic acid obtained from Sigma Chemical Company (St. Louis, Mo., catalog number C-2875). One hundred milliliters of 0.06 M sodium acetate, pH 4.8, was added to 1 L of pooled goat serum. The mixture was stirred for 15 minutes and the pH was adjusted to 4.8±0.15 by drop-wise addition of glacial acetic acid. Sixty milliliters of caprylic acid was added dropwise to the mixture. During the addition of the caprylic acid, the stirring was vigorous, but not vigorous enough to cause foaming. After the addition of the caprylic acid, the stirring was continued for overnight at 4° C. The mixture was centrifuged at 10,000×g for 30 minutes. The supernatant was poured through a metal strainer to remove protein flocculant and was subsequently filtered through a 1-μm filter. The filtrate was dialyzed against 10 volumes of PBS buffer at 2-8° C. and stored at 4° C.

### C. Preparation of a PBP2a Affinity Column.

The affinity antigen prepared in Example 2A was coupled to a NHS activated Sepharose columns described in Example 1B.

### D. Chromatography Conditions

All chromatography conditions, including the equipment, mobile phase solvents, and the timing of the mobile phase transitions during the chromatography were similar to those described in Example 1C.

### E. ELISA Assay for Anti-PBP2a Antibody Activity.

Pooled sera and eluent fractions from the affinity column were screened for the presence of anti-PBP2a antibodies using procedures similar to those described in Example 1D, with the exception that the PBP2a antigen was used to coat the wells of the plates.

Serial two-fold dilutions of the anti-PBP2a rabbit serum samples (or affinity column fractions), diluted in PBBT (PBST containing 0.1% Bovine Serum Albumin) were added to duplicate wells in the plates. The plates were processed as described for the anti-Clf40 assay in Example 1. When the ELISA reaction was complete (after 15 minutes at room temperature), the absorbance at 405 nm (A₄₀₅) of each well was measured using a plate reader. The titer of a sample was determined to be the highest dilution that gave a positive response (A₄₀₅>0.1 absorbance units) in the ELISA assay.

### F. Affinity Purification of Anti-PBP2a Antibody

In this example, pooled samples from the eluted, affinity-purified antibody were tested for anti-PBP2a activity according to the procedure described in Example 2E. The results of the ELISA testing from three purification experiments, representative of serum from three separate serum samples, are shown in Table 5. The data show that the selective elution process yielded a significant enrichment of anti-PBP2a specific antibody activity in all three cases.

TABLE 5
Anti-PBP2a antibody titers from chromatography eluate fractions.
Fraction buffers A, B, and C correspond to the column flow-through,
the sodium acetate elution buffer, and the citric acid elution buffer,
respectively. All antibody titers are calculated as the average of at
least two duplicate wells in the ELISA assay and are reported in μg/mL,
as described in Example 2E.

| Fraction | | | |
|---|---|---|---|
| Buffer | 1 | 2 | 3 |
| A | 1.25 | 2.5 | 0.625 |
| B | 0.039 | 0.078 | 0.156 |
| C | <0.019 | 0.039 | 0.039 |

### Example 3

### Optimization of Concentrations of Capture and Capping Antibodies Used in the Tandem Protein A/Clumping Factor Enzyme Linked Immunosorbant Assay (ELISA)

### A. General Materials and Methods

The antibodies used in the assay included two anti-Staphylococcus aureus protein A monoclonal antibodies (MAb-76, described in U.S. patent application Ser. No. 11/562,759, and MAb-107, described in U.S. patent application Ser. No. 11/562,747), the anti-Staphylococcus aureus clumping factor monoclonal antibody, MAb 12-9 (Inhibitex, Inc., Alpharetta, Ga., described in U.S. Pat. No. 6,177,084), and RxClf40 (rabbit anti-Clf40 antisera, Inhibitex, Inc.). Prior to use in the ELISA assays, the RxClf40 antibody was affinity purified according to the process described above. All capping antibodies were biotinylated prior to use. Antibodies were biotinylated according to the manufacturer's instructions using the EZ-LINK NHS-PEO4-Biotin kit from Pierce (Rockford, Ill.).

Phosphate buffered saline (PBS, 137 mM NaCl and 2.7 mM KCl in 10 mM phosphate buffer, pH 7.50) used in these experiments was prepared from a 10× concentrated solution obtained from EMD Biosciences, Inc. (San Diego, Calif.). The PBST reagent was prepared by adding 0.05% (v/v) TWEEN 20 to the PBS buffer. Costar 96-well high-binding polystyrene microtiter plates were obtained from Corning Inc. Life Sciences (Lowell, Mass.). All buffers were filtered prior to use except the wash buffer. All procedures were performed at room temperature unless specified otherwise. All ELISA wash procedures included five sequential wash volumes of 200 microliters per well and all washes were done with PBST buffer. Alkaline phosphatase chromogenic substrate, pNPP, was obtained from KPL, Inc. (Gaithersburg, Md.).

Antigens used in the optimization experiments included Protein A antigen from Zymed Laboratories (Invitrogen, Inc., Carlsbad, Calif.), clumping factor antigen (rClf40, Inhibitex, Inc., Alpharetta, Ga.) and lysed cells of S. aureus strain 25923. S. aureus 25923 was obtained from the American Type Culture Collection (Manassas, Va.). All antigens were prepared in filtered lysing solution containing lysostaphin (Sigma Aldrich, St. Louis, Mo.) diluted to 3 μg/mL in PBS containing 0.2% w/v PLURONIC L64 (BASF, Florham Park, N.J.) and 50 mM disodium EDTA, pH 7.44.

To prepare the S. aureus antigen, strain 25923 was grown at 37° C. in tryptic soy broth (Hardy Diagnostics, Santa Maria, Calif.) overnight to an estimated concentration of 5.0×10⁸ cfu/mL. The culture was washed twice (centrifuged at 4° C. for 10 minutes at 10,000 rpm, then resuspended in PBSL (PBS containing 0.2% w/v PLURONIC L64, BASF, Florham Park, N.J.). The washed bacterial stock was diluted 1:50 in PBSL to an approximate concentration of 1.0×10⁷ cfu/mL. This was further diluted 1:100 into lysing solution, for a final sample concentration of 1.0×10⁵ cfu/mL. Unlysed, diluted bacteria were plated on blood agar plates to verify the concentration of the original broth cultures. Assay results reflect the actual starting bacterial concentration.

### B. ELISA Assay Conditions

One or two capture antibodies were used in the assay. The antibodies were diluted from their refrigerated (4° C.) storage starting concentrations to microwell coating concentrations in PBS. One hundred microliters of the coating solutions were added to the wells of the microwell plates. Plates were incubated at 37° C. for 60 minutes. The coating solutions were removed prior to the blocking step.

All microtiter plates were blocked with 200 μL/well of STABILCOAT immunoassay stabilizer (Surmodics, Inc., Eden Prairie, Minn.), then incubated overnight at 4° C. The blocking solution was removed prior to the antigen coating step.

One-hundred microliters of the appropriate solution of capture antigens were added to each well and the plates were incubated for 60 minutes at 37° C. The plates were washed subsequently with PBST solution.

Two capping antibodies were used in the assay. All capping antibody preparations were diluted in Blotto (2% dried nonfat milk in PBST). One hundred microliters of the appropriate capping antibody mixture was added to each well and the plates were incubated for 60 minutes at 37° C.

Streptavidin-conjugated alkaline phosphatase (Streptavidin AP, Jackson Immunoresearch Laboratories, Inc., West Grove, Pa.) was diluted in blotto to a concentration of 0.5 ng/mL. The microtiter plates were washed, and 100 μL/well of the Streptavidin AP was added to the plates. The plates were incubated for 60 minutes at 37° C.

Plates were washed. Subsequently, 100 μL/well of pNPP substrate was added to the plates. The plates were held at room temperature for 15 minutes to observe the development of color. The alkaline phosphatase reaction was stopped by adding 100 μL/well of 5% (w/v) disodium EDTA and the plates were placed in a plate reader, where the absorbance at 405 nm was read.

### C. Optimization of Capture Antibody Concentrations and Ratios for the Detection of Staphylococcal Antigens.

ELISA assays were set up as described above. The objective of this study was to determine which concentrations of capture antibodies and capping antibodies provide the best binding (highest absorbance reading in the ELISA assay) with all three antigen preparations and, concomitantly, provide the lowest background (lowest absorbance reading in the ELISA assay). Seven 96-well plates were coated with the capture antibody combinations shown in Table 6.

TABLE 6
Capture antibody concentrations used in optimization study.
All concentrations are listed in μg/mL.

| | | MAb 12-9 |
|---|---|---|
| Column | MAb-76 | anti-clumping |
| Number | anti-protein A | factor |
| 1 | 7.5 | 0 |
| 2 | 7.5 | 7.5 |
| 3 | 3.75 | 7.5 |
| 4 | 1.88 | 7.5 |
| 5 | 0.93 | 7.5 |
| 6 | 0.47 | 7.5 |
| 7 | 0 | 7.5 |
| 8 | 3.5 | 3.5 |
| 9 | 7.5 | 3.75 |
| 10 | 7.5 | 1.88 |
| 11 | 7.5 | 0.93 |
| 12 | 7.5 | 0.47 |

Four antigens were run in the assay: 1) PBS (no antigen control); 2) rClf40 clumping factor protein diluted in lysing solution to 1 ng/mL; 3) Zymed Protein A diluted in lysing solution to 100 pg/mL; and 4) S. aureus strain 25923 (ATCC, Manassas, Va.), prepared as described above. One hundred microliter aliquots of antigen were added to the plates and incubated as described above.

Seven tandem capping antibody mixtures were made, one mixture for each microtiter plate. The capping antibody mixtures are shown in Table 7.

TABLE 7
Capping antibody concentrations used in optimization study.
All concentrations are listed in μg/mL.

| | | Rabbit anti- |
|---|---|---|
| | MAb-107.biotin | clumping |
| Plate Number | anti-protein A | factor.biotin |
| 1 | 2.5 | 0 |
| 2 | 0 | 2.5 |
| 3 | 2.5 | 2.5 |
| 4 | 1.25 | 2.5 |
| 5 | 2.5 | 1.25 |
| 6 | 2.5 | 0.625 |
| 7 | 0.625 | 2.5 |

Table 8 shows representative ELISA results from one of the plates that were used in the study. The data from all of the experiments indicated that the combination of capture antibodies concentrations consisting of 0.93 μg/mL MAb-76 and 7.5 μg/mL MAb 12-9 gave the highest binding activities for the antigens and the lowest background readings for the PBS controls. This combination was selected for further optimization of the capping antibody mixture.

TABLE 8
ELISA assay results for the detection of Staphylococcal Clumping
factor, Protein A, and lysed Staphyloccus aureus antigens.
Capping Antibodies = 2.5 μg/mL RxClf40 + 2.5 μg/mL MAb-107

| | [MAb-76] | | | | | | | | | | | |
|---|---|---|---|---|---|---|---|---|---|---|---|---|
| | 7.5 | 7.5 | 3.75 | 1.88 | 0.93 | 0.47 | 0 | 3.5 | 7.5 | 7.5 | 7.5 | 7.5 |
| [MAb 12-9] | 0 | 7.5 | 7.5 | 7.5 | 7.5 | 7.5 | 7.5 | 3.5 | 3.75 | 1.88 | 0.93 | 0.47 |
| 0 | 0.344 | 0.261 | 0.179 | 0.144 | 0.120 | 0.108 | 0.095 | 0.178 | 0.277 | 0.279 | 0.310 | 0.281 |
| ClfA | 0.421 | 2.566 | 2.416 | 2.268 | 2.197 | 2.164 | 2.203 | 2.806 | 2.240 | 1.752 | 1.323 | 0.944 |
| ProtA | 1.032 | 0.917 | 0.815 | 0.701 | 0.634 | 0.582 | 0.116 | 0.825 | 0.886 | 0.896 | 0.939 | 0.930 |
| 25923 | 3.905 | 3.887 | 3.908 | 3.723 | 3.603 | 3.233 | 0.232 | 3.852 | 3.844 | 3.819 | 3.894 | 3.859 |

The capping antibodies in this study were biotin labeled affinity purified rabbit anti-clumping factor and biotin labeled MAb-107, both at a concentration of 2.5 μg/mL. The concentrations of each capture antibody (MAb-76 and Mab 12-9, respectively) in the various combinations are shown in μg/mL. The data are the average absorbance readings at 405 nm for a minimum of two duplicate wells. This table is representative data from a number of experiments that were done. Each experiment involved the use of different concentrations of capping antibodies.

### D. Optimization of Capping Antibody Concentrations for the Detection of Staphylococcal Antigens.

This experiment was conducted similar to the one described in Example 3C except that the capture antibody concentrations were held constant at 1 μg/mL (MAb-76) and 7.5 μg/mL (MAb 12-9). Representative data from these experiments are shown in Table 9. The data indicate that a mixture of capping antibodies consisting of MAb-107 at 2.5 μg/mL and affinity-purified RxClf40 at 0.75 μg/mL resulted in the detection of the lowest amounts of staphylococcal antigens.

TABLE 9
ELISA assay results for the detection of Staphylococcal Clumping
factor antigen. The concentrations of each capping antibody are
reported in μg/mL. The concentration of the antigen, Staphylococcal
clumping factor, in each test is reported in pg/mL. The data are the
average absorbance readings at 405 nm for a minimum of at
least two duplicate wells.

| | | Mab 107 (μg/mL) | | | |
|---|---|---|---|---|---|
| | | 2.5 | 2.5 | 2.5 | 2.5 |
| | | RxClf (μg/mL) | | | |
| | | 0.75 | 0.625 | 0.5 | 0.375 |
| ClfA | 250.00 | 0.700 | 0.661 | 0.593 | 0.483 |
| (pg/ml) | 125.00 | 0.388 | 0.360 | 0.320 | 0.273 |
| | 62.50 | 0.233 | 0.209 | 0.182 | 0.172 |
| | 31.25 | 0.152 | 0.144 | 0.131 | 0.107 |
| | 15.63 | 0.111 | 0.105 | 0.089 | 0.078 |
| | 7.81 | 0.089 | 0.079 | 0.068 | 0.062 |
| | 3.91 | 0.083 | 0.074 | 0.069 | 0.060 |
| | 0.00 | 0.071 | 0.075 | 0.068 | 0.058 |

The combination of antibody concentrations selected to do limit of detection studies is shown in Table 10.

TABLE 10
Optimal antibody combinations for the detection of staphylococcal
antigens in a sample using an ELISA test. The optimal amounts of
each antibody and the ratios for the respective capture and capping
antibodies were determined from experiments using various
combinations of antibody amounts and ratios. The criteria for
selection of the optimal amount included the largest detection signal
for each antigen, coupled with the lowest background signal when
no antigen was present in the assay.

| | | | MAb- | Affinity-purified |
|---|---|---|---|---|
| | MAb-76 | MAb 12.9 | 107.biotin | RxClf40.biotin |
| Coating | 1 μg/mL | 7.5 μg/mL | N/A | N/A |
| concentration | | | | |
| Capping | N/A | N/A | 2.5 μg/mL | 0.75 μg/mL |
| concentration | | | | |

### Example 4

### Determination of Lower Limit of Detection for Protein A and Clumping Factor Antigens in the Tandem Protein A/Clumping Factor Enzyme Linked Immunosorbant Assay (ELISA)

All procedures were performed as described in Example 3. Antigens were prepared in filtered lysing solution containing lysostaphin (Sigma Aldrich, St. Louis, Mo.) diluted to 3 μg/mL in PBS containing 0.2% w/v PLURONIC L64 (BASF, Florham Park, N.J.) and 50 mM disodium EDTA, pH 7.44.

Three antigens were run in the assay: 1) Clf40 clumping factor protein diluted by serial two-fold dilutions from 2 ng/mL to 0.0019 ng/mL; 2) Zymed Protein A (SpA) diluted by serial two-fold dilutions from 250 pg/mL to 0.24 pg/mL; and 3) S. aureus strain 25923, prepared as in Example 1, except that it was diluted in serial two-fold dilutions to final sample concentrations of approximately 5×10⁵ CFU/mL to approximately 4.8×10² CFU/mL. A control containing only lysing solution as described above, without antigen, was also included on the plates.

The lower limit of detection for each antigen was determined by choosing the sample concentration values that were three standard deviations above the control, with continually increasing absorbance values at that concentration point and above. For the tandem assay, the lower limit of detection for clumping factor antigen was less than or equal to 0.0019 ng/mL, and the lower limit of detection for protein A was 7.81 pg/mL. The lower limit of detection for lysed S. aureus strain 25923 was 3,828 cells/mL. The results of this study are shown in Table 11.

TABLE 11
Detection of staphylococcal antigens in an ELISA assay. The values
shown for the Tandem assays are the average absorbance (405 nm)
readings from a minimum of at least two duplicate wells.
Capture Antibodies: Mab 12-9 (7.5 μg/mL) + MAb-76 (1 μg/mL)

| Clumping Factor | | Protein A | | Lysed S. aureus | |
|---|---|---|---|---|---|
| Clf | | SpA | | S. aureus | |
| (ng/mL) | Tandem | (pg/mL) | Tandem | (cfu/mL) | Tandem |
| 2.00000 | 4.679 | 250.00000 | 1.653 | 490,000 | 5.000 |
| 1.00000 | 3.022 | 125.00000 | 0.943 | 245,000 | 5.000 |
| 0.50000 | 1.789 | 62.50000 | 0.620 | 122,500 | 5.000 |
| 0.25000 | 1.065 | 31.25000 | 0.350 | 61,250 | 3.831 |
| 0.12500 | 0.646 | 15.62500 | 0.237 | 30,625 | 2.475 |
| 0.06250 | 0.430 | 7.81250 | 0.230 | 15,313 | 1.496 |
| 0.03125 | 0.321 | 3.90625 | 0.127 | 7,656 | 0.855 |
| 0.01563 | 0.276 | 1.95312 | 0.132 | 3,828 | 0.493 |
| 0.00781 | 0.247 | 0.97656 | 0.135 | 1,914 | 0.313 |
| 0.00391 | 0.242 | 0.48828 | 0.116 | 957 | 0.233 |
| 0.00195 | 0.234 | 0.24414 | 0.115 | 479 | 0.177 |
| 0.00000 | 0.221 | 0.00000 | 0.117 | 0 | 0.196 |
| 0 + 3 SD | 0.221 | 0 + 3 SD | 0.136 | 0 + 3 SD | 0.435 |

### Example 5

### Purification of High-Affinity Anti-Clumping Factor from Rabbit Antiserum

Pooled rabbit serum was obtained as described in Example 1A. The preparation of the Clf40 affinity column was as described in Example 1B. Chromatography was performed as described in Example 1C, except that the buffers used were those listed in Table 12, below.

TABLE 12

| SOLVENT | COMPOSITION |
|---|---|
| Binding Buffer A | Phosphate Buffered Saline, pH 7.3 |
| Elution Buffer B | 20 mM Sodium Acetate, 0.5M NaCl, pH 4.0 |
| Elution Buffer C1 | 0.1M Citric Acid, pH 2.1 |
| Elution Buffer C2 | 0.1M Citric Acid, pH 3.0 |
| Elution Buffer C3 | 6.7 mM Citric Acid, 6.7 mM Orthophosphoric |
| | Acid, 11.4 mM Orthoboric Acid, 20% |
| | Acetonitrile, pH 2.0 |
| Elution Buffer C4 | 6.7 mM Citric Acid, 6.7 mM Orthophosphoric |
| | Acid, 11.4 mM Orthoboric Acid, 20% |
| | Acetonitrile, pH 3.0 |

All four elution buffers generated similar elution profiles.

The avidity of each eluted antibody fraction was determined by ELISA analysis performed as described in Example 1D with the following changes: the plate was blocked with STABILCOAT (SurModics, Inc., Eden Prairie, Minn.) overnight at 4° C., and the rabbit serum was diluted (serial two-fold dilutions) in PBST+0.5% bovine serum albumin starting at 10 ng/mL.

Results are reported in Table 13. Avidity is reported as the minimum concentration of antibody to generate a positive response (i.e., three standard deviations over background) and is expressed as nanograms per milliliter (ng/mL).

TABLE 13

| Fraction | Elution Buffer | Column Load | Avidity (ng/mL) |
|---|---|---|---|
| F1 | C1 | 15 mg | 1.25 |
| F2 | C1 | 30 mg | 0.625 |
| F3 | C2 | 30 mg | 0.625 |
| F4 | C4 | 30 mg | 1.25 |
| F5 | C3 | 24 mg | 1.25 |
| F6 | C3 | 24 mg | 1.25 |
| Pos Ctrl^{a} | | | 0.313 |
| Neg Ctrl^{b} | | | 0 |

^{a}positive control, Clf40 AP Anti-Clf 40 Ab
^{b}negative control, rabbit IgG

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

For any method disclosed herein that includes discrete steps, the steps may be conducted in any feasible order. And, as appropriate, any combination of two or more steps may be conducted simultaneously.

What is claimed is:

1. An anti-Staphylococcus aureus clumping factor protein polyclonal antibody preparation having an avidity expressed as an endpoint concentration of at least 0.01 ng/mL and no greater than 15 ng/mL.

2. The anti-Staphylococcus aureus clumping factor protein polyclonal antibody preparation of claim 1 wherein the avidity is expressed as an endpoint concentration of at least 0.625 ng/mL.

3. The anti-Staphylococcus aureus clumping factor protein polyclonal antibody preparation of claim 1 wherein the avidity is expressed as an endpoint concentration of at least 1.0 ng/mL.

4. The anti-Staphylococcus aureus clumping factor protein polyclonal antibody preparation of claim 1 wherein the avidity is expressed as an endpoint concentration of at least 1.25 ng/mL.

5. The anti-Staphylococcus aureus clumping factor protein polyclonal antibody preparation of claim 1 wherein the avidity is expressed as an endpoint concentration of at least 2.0 ng/mL.

6. The anti-Staphylococcus aureus clumping factor protein polyclonal antibody preparation of claim 1 wherein the avidity is expressed as an endpoint concentration of at least 3.9 ng/mL.

7. The anti-Staphylococcus aureus clumping factor protein polyclonal antibody preparation of claim 1 wherein the avidity is expressed as an endpoint concentration of no greater than 7.8 ng/mL.

8. The anti-Staphylococcus aureus clumping factor protein polyclonal antibody preparation of claim 1 wherein the avidity is expressed as an endpoint concentration of no greater than 3.9 ng/mL.

9. The anti-Staphylococcus aureus clumping factor protein polyclonal antibody preparation of claim 1 wherein the avidity is expressed as an endpoint concentration of no greater than 2.0 ng/mL.

10. A high avidity anti-Staphylococcus aureus clumping factor protein polyclonal antibody preparation, wherein the high avidity anti-S. aureus clumping factor protein polyclonal antibody preparation detects recombinant clumping factor (rClf40) protein of S. aureus at a concentration of about 1 to about 100 picogram per milliliter (pg/mL).

11. A high avidity anti-Staphylococcus aureus clumping factor protein polyclonal antibody preparation, wherein the high avidity anti-S. aureus clumping factor protein polyclonal antibody preparation demonstrates at least a 4-fold increase in avidity as measured by endpoint concentration in comparison to a Staphylococcus aureus clumping factor protein antiserum.

12. A high avidity anti-Staphylococcus aureus clumping factor protein polyclonal antibody preparation, wherein the high avidity anti-S. aureus clumping factor protein polyclonal antibody preparation is prepared by a method comprising:
obtaining antiserum from an animal immunized with recombinant clumping factor (rClf40) protein of S. aureus;
binding the antiserum to a S. aureus clumping factor (Clf40) protein affinity column;
washing the column with a wash buffer comprising about 0.5 M salt and a pH of about 4; and
eluting the high avidity anti-S. aureus clumping factor protein polyclonal antibody preparation from the column with an elution buffer with a pH of about 2.

13. The high avidity anti-Staphylococcus aureus clumping factor protein polyclonal antibody preparation of claim 12, wherein the method further comprises enriching the antiserum for the IgG class of antibodies prior to binding the antiserum to a S. aureus clumping factor (Clf40) protein affinity column.

14. The high avidity anti-Staphylococcus aureus clumping factor protein polyclonal antibody preparation of claim 13, wherein enriching for the IgG class of antibodies is by Protein A binding.

15. The high avidity anti-Staphylococcus aureus clumping factor protein polyclonal antibody preparation of claim 12, wherein the binding, washing, and/or eluting take place in the absence of acetonitrile.

16. A high avidity anti-Staphylococcus aureus clumping factor protein polyclonal antibody preparation, wherein the high avidity anti-S. aureus clumping factor protein polyclonal antibody preparation binds to recombinant clumping factor (rClf40) protein of S. aureus with antigen binding activity similar to that of fraction C1 or C2 of experiment B; fraction C of experiment C; fraction C of experiment D; fraction C of experiment E; fraction C of experiment F; fraction C of experiment G; or fraction C of experiment H, as shown in Table 4.

17. A high avidity anti-penicillin-binding protein 2a (PBP2a) polyclonal antibody preparation, wherein the high avidity anti-S. aureus clumping factor protein polyclonal antibody preparation demonstrates at least a 2-fold increase in avidity as measured by endpoint concentration in comparison to a Staphylococcus aureus clumping factor protein antiserum.

18. A method of preparing a high avidity anti-Staphylococcus aureus clumping factor protein polyclonal antibody preparation, the method comprising:
obtaining antiserum from an animal immunized with recombinant clumping factor (rClf40) protein of S. aureus;
binding the antiserum to a S. aureus clumping factor (Clf40) protein affinity column;
washing the column with a wash buffer comprising about 0.5 M salt and a pH of about 4; and
eluting the high avidity anti-S. aureus clumping factor protein polyclonal antibody preparation from the column with an elution buffer with a pH of about 2.

19. The method of claim 18, the method further comprising enriching the antiserum for the IgG class of antibodies prior to binding the antiserum to the antigen affinity column.

20. The method of claim 18, wherein enriching for the IgG class of antibodies is by Protein A binding.

21. The method of claim 18, wherein the binding, washing, and/or eluting take place in the absence of acetonitrile.

22. A method of preparing a high avidity anti-penicillin-binding protein 2a (PBP2a) polyclonal antibody preparation, the method comprising:
obtaining antiserum from an animal immunized with PBP2a;
binding the antiserum to a PBP2a affinity column;
washing the column with a wash buffer comprising about 0.5 M salt and a pH of about 4; and
eluting the high avidity anti-PBP2a polyclonal antibody preparation from the column with an elution buffer with a pH of about 2.

23. The method of claim 22, the method further comprising enriching the antiserum for the IgG class of antibodies prior to binding the antiserum to the antigen affinity column.

24. The method of claim 22, wherein enriching for the IgG class of antibodies is by Protein A binding.

25. The method of claim 22, wherein the binding, washing, and/or eluting take place in the absence of acetonitrile.

26. A method of preparing a high avidity anti-antigen polyclonal antibody preparation, the method comprising:
obtaining an antiserum from an animal immunized with the antigen;
binding the antiserum to an antigen affinity column;
washing the column with a wash buffer comprising about 0.5M salt and a pH of about 4; and
eluting the high avidity anti-antigen polyclonal antibody preparation from the column with an elution buffer with a pH of about 2.

27. The method of claim 26, the method further comprising enriching the antiserum for the IgG class of antibodies prior to binding the antiserum to the antigen affinity column.

28. The method of claim 26, wherein enriching for the IgG class of antibodies is by Protein A binding.

29. The method of claim 26, wherein the binding, washing, and/or eluting take place in the absence of acetonitrile.


SEQUENCE LISTING (1 sequence)

SEQ ID NO: 1 - DNA, 2061 bp, Staphylococcus aureus
aataattttgtttaactttaaggaggtattaattaaatgaaacaccatcaccatcatcactatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacagcatatgagataggcatcgttccaaagaatgtatctaaaaaagattataaagcaatcgctaaagaactaagtatttctgaagactatatcaaacaacaaatggatcaaaattgggtacaagatgataccttcgttccacttaaaaccgttaaaaaaatggatgaatatttaagtgatttcgcaaaaaaatttcatcttacaactaatgaaacagaaagtcgtaactatcctctaggaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaactgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataatgaggtaccctggtgccacgcggttccatggcgatatcggatccgaattcgagctccgtc